(19) United States
(12) Patent Application Publication
(10) Pub. No.: US 2015/0031940 A1
(43) Pub. Date: Jan. 29, 2015

(54) Enunciation Device

(71) Applicant: Daniel Floyd, OLYMPIA FIELDS, IL (US)

(72) Inventor: Daniel Floyd, OLYMPIA FIELDS, IL (US)

(21) Appl. No.: 14/511,961

(22) Filed: Oct. 10, 2014

Related U.S. Application Data
(63) Continuation-in-part of application No. 13/683,164, filed on Nov. 21, 2012.
(60) Provisional application No. 61/632,368, filed on Jan. 23, 2012.

(51) Int. Cl. A61F 5/58

(52) U.S. Cl. CPC A61F 5/58
USPC 600/24

(57) ABSTRACT

This invention relates an enunciation device comprising a device, preferably hollowed, shaped in a manner to fit a mouth. This enunciation device assists with improving enunciation, slowing speech, and strengthens mouth, tongue, and jaw muscles so that a user can build confidence while speaking.

### CROSS-REFERENCE TO RELATED APPLICATIONS

This application is a continuation in part application that relates to and claims priority from the earlier filed U.S. non-provisional application Ser. No. 13/683,164 filed on Nov. 21, 2012 having the title “Enunciation Device”. U.S. application Ser. No. 13/683,164 claims priority to an earlier filed United States provisional application No. 61/632,368 filed on Jan. 23, 2012 having the title “Pronunciation device: Is a one-component device that individuals can use to improve their articulation abilities. The more the device is used the stronger the overall mouth muscles and the better the individual's speech”.

### STATEMENT REGARDING FEDERALLY SPONSORED RESEARCH OR DEVELOPMENT

Not applicable.

### REFERENCE TO SEQUENCE LISTING, A TABLE, OR A COMPUTER PROGRAM LIST COMPACT DISC APPENDIX

Not applicable.

### BACKGROUND OF THE INVENTION

Proper pronunciation and enunciation of words while speaking is an important skill a person should develop. Being heard clearly and having proper articulation of words is a skill that all people need to be successful communicators. For some this skill comes easier than it does to others. As such, there is a desire for a device that helps build mouth, tongue, and jaw muscles while slowing speech and improving enunciation of words so that a user can speak confidently and clearly.

It is known in prior art that there are exercises and relaxation techniques designed to stretch and work the mouth so that better speech can be achieved. Historically, actors or public speakers have used items such as a pencil or a cork held in the mouth while practicing speaking that the user is forced to work muscles that may not be normally used in the hopes that improved speech by way of stronger muscles will result. However, this is a rudimentary manner of developing good enunciation skills and there is a demand for a more sophisticated device.

Other more sophisticated devices have been developed, such as the one seen in U.S. Pat. No. 1,869,391 (Quintina). This device relates to control of vocal tones. This device is placed into the mouth and pushes the tongue downward to teach a user to open the mouth more widely. This device is useful in teaching a user to open the mouth in order to speak more clearly but is only designed to perform that function (holding the tongue down) and does not allow a user to fully exercise the mouth.

Another such device is seen in U.S. Pat. No. 3,014,286 (Hricak). This relates to a guide to language study whereby a student is trying to learn a new language that is foreign to the student's native language and accompanying sounds, such as an English speaker learning French or vice versa. This device is placed into the mouth of the language student to assist in controlling placement and shape of the mouth, teeth, and tongue. This device is problematic for someone looking to enunciate more clearly needs as it impedes the lips from being able to push outward. This device has a wall-like barrier holding the lips in that impedes such movement.

Another device is seen in U.S. Pat. No. 3,286, 576 (West). This patent relates to exercising the lips for speaking or playing wind instruments. The lip movement while playing a wind instrument is particularly important and this device helps to control that movement. However, this device also does not provide a more complete approach to exercising the lips, mouth, tongue and accompanying muscles so that enunciation can be improved .

Other devices such as those seen in U.S. Pat. No. 5,257,930 (Blakeley) or U.S. Pat. No. 7,214,064 (Hall) are small devices placed in distinct parts of the mouth to assist with certain distinct sounds such as the “r” sound.

The problem with all these speech aid devices is that none provide a full mouth exercise to slow a users speech to enable them to learn to speak more slowly and clearly. This invention builds on the known prior art to provide for a device that allows for free movement of lips, slowed movement of the tongue, while holding the mouth open so that speech can be practiced in a more articulate manner.

This invention in its most basic form can be viewed as a hands free mouth resistance exercise device, similar to weighted boxing gloves and other devices built for resistance related exercises. Looking at weighted boxing gloves as an example, the weighted gloves are challenging to a boxer in that every muscle in his arm works harder to accommodate the added weight and resistance. Slowing down the speed in which the boxer punches and forces the arm muscle to stretch. Once the weighted gloves are removed the boxer's arm is strengthened which improves the boxers punching skill and technique. This same concept is applied to this invention. When the device is used it is challenging the mouth by overworking the quadrates labbii superioris, caninus, zygomaticus, mentalis, quadratus labbii inferiors, triangulars, buccinator, orbicularis oris, risorius and the tongue. These muscles are working harder with the invention to properly enunciate words and sounds so that it is easier without the invention.

The concept and functionality of this device is to challenge and improve a users enunciation skills by resistance from a foreign object (the invention) to create better enunciation, strengthening facial muscles to slow down speaking pace and build confidence. This device will be very beneficial to public speakers and children and adults that suffer from speech disorders.

The above references to and descriptions of prior proposals or products are not intended to be, and are not to be construed as, statements or admissions of common general knowledge in the art. In particular, the above prior art discussion does not relate to what is commonly or well known by the person skilled in the art, but assists in the understanding of the inventive step of the present invention of which the identification of pertinent prior art proposals is but one part.

### BRIEF SUMMARY OF THE INVENTION

This invention relates to an enunciation device. More specifically it relates to an enunciation device that comprises a device shaped to comfortably fit a human mouth so that when a user speaks with it in the mouth, the muscles of the mouth, jaw, and tongue are exercised to result in clearer, more articulate, and more enunciated speech. This hollow device comprises a plastic, silicone or rubber material that may be transparent or opaque in nature. One skilled in the art will recognize that other soft, semi-rigid materials may be suitable.

The enunciation device is preferably hollowed so that when a user places it inside the mouth and begins to speak, the words can be heard clearly. One skilled in the art will recognize that the device does not necessarily need to be hollowed but it is preferred that it is hollowed. Further, an important aspect of proper enunciation and articulation of words is the control of one's breath. The device allows also user to be able to breathe through the hollowed portion as needed and serves to reduce incidence of drooling and saliva can pass through the hallowed portion. Further, in the extremely unlikely event that the device becomes lodged in a users throat, the hollowed portion would allow a user to breathe through the holes. The hollowed portion has a larger diameter opening at the first opening located at the front of the device than at the second opening located at the back of the device. The two openings are connected by a channel that allows air to pass through. The difference in diameter between the two openings is important as it forces a user to control breathing while using the device.

This device itself does not emit any audible sound. Any sound that is heard while using the device comes from the user of the device, not the device itself. The device is hollow to only let air pass through so that a user can control his/her breathing while using the device. Any sound from the device, whether audible to a human or not, would cause severe distraction to a user as the user must be only listening to his/her own voice or breathing.

The device is shaped in a manner that ergonomically fits a human mouth. While the device is generally cylindrical in shape, it does taper off into a projection or tail towards the roof of a mouth. This projection allows for more space in the mouth for the tongue to move while the enunciation device is in use. However, the tail does also prevent the tongue from touching the roof of the mouth. When the tongue is blocked from touching the roof of the mouth, it forces the user to over enunciate which is basic to the resistance exercise concept. The tail is also grooved to make the device more flexible and comfortable.

Surrounding the diameter of the device is a tooth ring. This tooth ring is of smaller diameter then the hollow device. This tooth ring serves as a guide for proper placement in the mouth as the user bites down on the device around the tooth ring in order to properly hold the enunciation device in place.

On the top of the device, there is a groove located on top of the tail. This groove makes the tail flexible so when the tongue hits the tail it has some “give” to provide a user with a more comfortable fit. Some embodiments may include a cap that fits around the tooth ring. This cap allows a user to cover the portion of the enunciation device that is placed in the mouth for a more hygienic setting while not in use.

Some embodiments may also include a lanyard loop extending off the portion of the hollow device that is not placed in the mouth.

This and other embodiments will be more thoroughly realized in the drawings and detailed description below.

### BRIEF DESCRIPTION OF THE SEVERAL VIEWS OF THE DRAWINGS

Possible and preferred features of the present invention will now be described with particular reference to the accompanying drawings. However, it is to be understood that the features illustrated in and described with reference to the drawings are not to be construed as limiting on the broad scope of the invention. In the drawings:

FIG. 1 is a top perspective view of the enunciation device.

FIG. 2 is a side perspective view of the enunciation device.

FIG. 3 is a side perspective view of the enunciation device including the cap.

FIG. 4 is a view through the enunciation device from the front end.

FIG. 5 is a view through the enunciation device from the back end.

FIG. 6 depicts the enunciation device within a user's mouth.

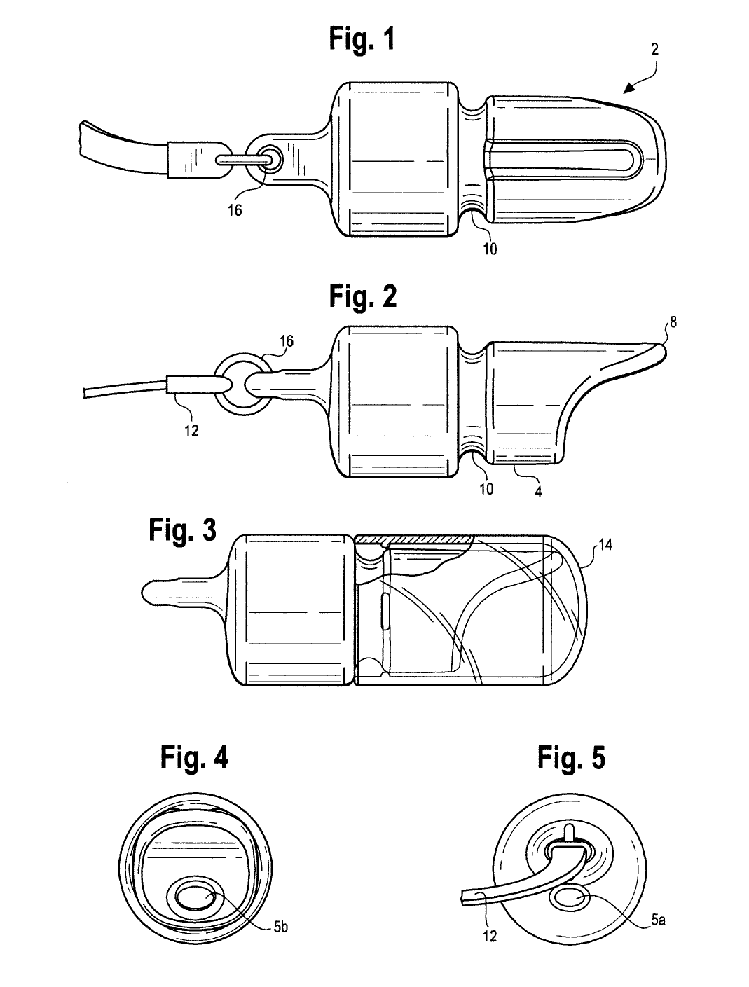

### DETAILED DESCRIPTION OF THE INVENTION

Turning to the Figures, this invention relates to an enunciation device 2 comprising a device 4 ergonomically shaped to fit within a mouth 6 tapering into a tail 8 towards the roof of a mouth 6. As seen in FIGS. 4 and 5, the hollow device 4 has a smaller diameter opening 5a at the back of the device 4 than the front of the device 5b. While the difference in diameter is not anticipated to be large, the smaller opening 5a at the back of the device is to allow for more control over one's breathing while using the hollow device 4. The small opening 5a is joined to the larger opening 5b by way of a channel 7. While the device is in use, air passes freely through the channel 7 without generating any audible sound.

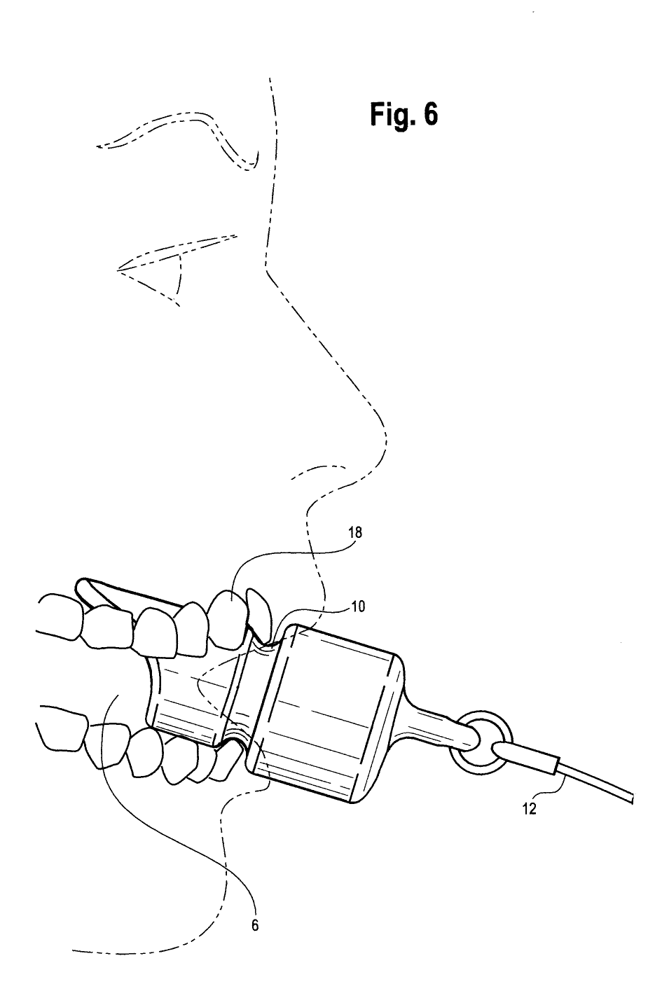

As seen in FIG. 6, when the device 4 is place in the mouth 6, the user's teeth 18 bite down around the tooth ring 10. This ensures proper placement of the device 4. The tail 8 of the device 4 rests towards the roof of the mouth 6.

FIG. 3 depicts a removable cap 14. This removable cap 14 fits over the device 4 and locks into place around the tooth ring 10. This cap 14 provides for a hygienic situation for the device 4 when not in use. If a user desires to use the device 4, then the cap 14 is to be removed.

The enunciation device 2 further comprises a ring 16 and lanyard 12. This allows a user to keep the device 4 around his or her neck or loop it in another convenient location so the hollow device 4 is not lost.

Throughout the specification and claims the word “comprise” and its derivatives are intended to have an inclusive rather than exclusive meaning unless the contrary is expressly stated or the context requires otherwise. That is, the word “comprise” and its derivatives will be taken to indicate the inclusion of not only the listed components, steps or features that it directly references, but also other components, steps or features not specifically listed, unless the contrary is expressly stated or the context requires otherwise.

It will be appreciated by those skilled in the art that many modifications and variations may be made to the methods of the invention described herein without departing from the spirit and scope of the invention.

What is claimed is:

1. An exercise device for a mouth, tongue, and jaw muscles comprising:
a device ergonomically shaped to fit within a mouth for resistance training of the mouth, tongue, and jaw; said device tapering into a tail towards the roof of a the mouth;
wherein the device further comprises a first opening located at the front of the device connected to a second opening located at back of the device by a channel; whereas said channel allows air to pass through without generating any audible sound from the device and the device provides for resistance training of the mouth, tongue, and jaw muscles.

2. The device as in claim 1 wherein the device has a larger diameter opening at the first opening located at the front of the device than the second opening located at the back of the device.

3. The device as in claim 1 further comprising a tooth ring.

4. The device as in claim 3 further comprising a removable cap that closes over a portion of the hollow device and locks onto said tooth ring.

5. The device of claim 1 further comprising a ring and lanyard loop.

6. The device of claim 1 wherein said device comprises plastic, silicone, or rubber.

7. The device of claim 1 wherein the device is hands free.
